(19) United States
(12) Patent Application Publication
(10) Pub. No.: US 2026/0014359 A1
(43) Pub. Date: Jan. 15, 2026

(54) MICRONEEDLE STRUCTURE

(71) Applicant: LINTEC CORPORATION, Tokyo (JP)

(72) Inventors: Yosuke KOMA, Tokyo (JP); Akio KABUTO, Tokyo (JP)

(21) Appl. No.: 18/880,051

(22) PCT Filed: Jun. 30, 2023
(86) PCT No.: PCT/JP2023/024316

§ 371 Date: Dec. 30, 2024

(30) Foreign Application Priority Data
Jun. 30, 2022 (JP) 2022-106672
Mar. 30, 2023 (WO) PCT/JP2023/013269

(51) Int. Cl. A61M 37/00; A61L 31/04; G01N 5/02

(52) U.S. Cl. CPC A61M 37/0015; A61L 31/042; G01N 5/02; A61M 2037/0023; A61M 2037/003; A61M 2205/70

(57) ABSTRACT

Microneedle structure of the present invention includes a needle-shaped portion having an interior formed with a hole portion. The needle-shaped portion contains a low-melting-point resin having a weight-average molecular weight of 25,000 or more and a melting point of 130° C. or lower. Such a microneedle structure can be configured to have a needle-shaped portion having high strength.

### TECHNICAL FIELD

The present invention relates to a microneedle structure.

### BACKGROUND ART

In recent years, there have been proposals for supplying a drug into the body or collecting body fluids from the body through through-holes formed in microneedles. For example, microneedles are known, which include a microneedle-shaped biocompatible matrix and porous particles provided on or at least partially in the biocompatible matrix (Patent Document 1).

### PRIOR ART DOCUMENTS

### Patent Documents

[Patent Document 1] JP2014-094171A

### SUMMARY OF THE INVENTION

### Problems to be Solved by the Invention

Patent Document 1 assumes that the microneedles may be absorbed in the body because biocompatible materials constituting the microneedles will swell or will be absorbed into biological tissue within a few seconds to a few hours when they are pierced into the skin. From the viewpoint of safety, however, it is desirable to remove the pierced microneedles so as not to leave them in the skin as much as possible. Here, if microneedles containing porous particles as disclosed, for example, in Patent Document 1 are pierced and then removed from the skin, there is a problem in that the microneedles may become defective due to poor strength. In addition, if the strength of needle-shaped portions of the microneedle structure is low, there is a possibility that the efficiency of supplying a drug or the like may be reduced due to breakage when the microneedles are pierced into the skin.

The present invention has been made in view of such actual circumstances, and an object of the present invention is to provide a microneedle structure having a needle-shaped portion with high strength.

### Means for Solving the Problems

To achieve the above object, first, the present invention provides a microneedle structure comprising a needle-shaped portion having an interior formed with a hole portion, the needle-shaped portion containing a low-melting-point resin having a weight-average molecular weight of 25,000 or more and a melting point of 130° C. or lower (Invention 1).

In the above invention (Invention 1), the needle-shaped portion contains a low-melting-point resin having a weight-average molecular weight of 25,000 or more and a melting point of 130° C. or lower, and can therefore maintain sufficient strength. That is, in the case of a structure in which a plurality of hole portions are opened on the side surface of the needle-shaped portion, it is possible to increase the rate of absorption or release of fluid from the needle-shaped portion as compared with a structure in which hole portions are opened only at the top portion of the needle-shaped portion, but it is conceivable that the needle-shaped portion may become brittle and the strength is insufficient. Fortunately, however, in the present invention, since the needle-shaped portion contains a low-melting-point resin having a weight-average molecular weight of 25,000 or more and a melting point of 130° C. or lower, the strength can be increased, and it is possible to suppress the breakage of the needle-shaped portion, for example, when piercing it into the skin.

In the above invention (Invention 1), the needle-shaped portion may preferably contain a water-insoluble hydrophilic resin (Invention 2).

In the above invention (Invention 1), the water-insoluble hydrophilic resin may be preferably a water-insoluble polysaccharide (Invention 3).

In the above invention or inventions (Invention or Inventions 1 to 3), the needle-shaped portion may be preferably formed with a porous structure (Invention 4).

In the above invention (Invention 4), preferably, the needle-shaped portion may have a base portion, and a water absorption ratio of the needle-shaped portion measured by a testing method below in a state in which the needle-shaped portion is composed only of the porous structure may be 8.5% or more (Invention 5).

### (Testing Method)

The needle-shaped portion is immersed in 10 ml of purified water under an environment of 25° C. The needle-shaped portion in an immersed state is placed under a reduced pressure environment of 0.09 MPa for 1 hour to allow water to penetrate into the interior of the porous structure. Subsequently, the needle-shaped portion is taken out from generation, and water droplets attached to the surface are removed. Water droplets on the surface of the needle-shaped portion on a side formed with a needle are removed by blowing them off with an air blow gun, and water droplets on the surface of the base portion side of the needle-shaped portion are removed through placing the base portion on a glass plate and allowing the needle-shaped portion's own weight to push the water droplets around the base portion, and after statically placing it for 5 seconds, the needle-shaped portion is picked up from the glass plate. Then, weight measurement is performed for a sample after water absorption. A water absorption ratio (ratio of absorbed water to the sample's own weight) is obtained using an equation below.

$$Water absorption ratio ( \% ) = ( weight of sample after water absorption - weight of sample before water absorption ) \div weight of sample before water absorption \times 100$$

In the above invention (Invention 1), the needle-shaped portion may preferably contain a filler (Invention 6).

### BRIEF DESCRIPTION OF DRAWINGS

FIG. 1 is a set of (1) a schematic cross-sectional diagram of a microneedle structure of the present invention and (2) a partial enlarged view of a needle-shaped portion.

FIG. 2 is a schematic partial cross-sectional diagram of a test patch using the microneedle structure of the present invention.

FIG. 3 is a set of explanatory diagrams (a) to (c) illustrating the procedure of a method for producing a microneedle structure according to an embodiment.

FIG. 4 is a set of explanatory diagrams (a) to (c) illustrating the procedure of a method for producing a microneedle structure according to an embodiment.

### EMBODIMENTS FOR CARRYING OUT THE INVENTION

Hereinafter, embodiments of the present invention will be described.

### <Microneedle Structure>

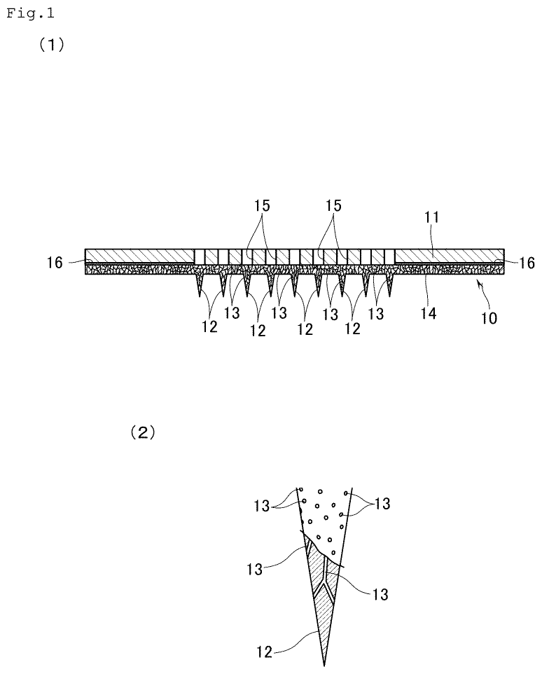

FIG. 1 illustrates a microneedle structure 10 according to an embodiment of the present invention. The microneedle structure 10 includes a plurality of needle-shaped portions 12 that are spaced apart from each other at predetermined intervals on one surface side of a base material 11. In addition, the needle-shaped portions 12 are each formed with a plurality of hole portions 13. The base material 11 is formed with through-holes 15. The microneedle structure 10 can be used as a test patch that absorbs a body fluid from inside the skin through the hole portions 13 of the needle-shaped portions 12 and performs a test using the body fluid obtained via the base material 11 or can also be used as a drug administration patch that administers a drug from the skin into the body via the base material 11 and the hole portions 13 of the needle-shaped portions 12. In the present invention, the body fluid refers to blood, lymph, interstitial fluid, etc.

### (1) Needle-Shaped Portions

The shape, size, formation pitch, and number of formation of the needle-shaped portions 12 can be appropriately selected depending on the intended use of the microneedles. Examples of the shape of the needle-shaped portions 12 include columnar, prismatic, conical, and pyramidal shapes. In the present embodiment, the shape of the needle-shaped portions 12 is pyramidal. The maximum diameter or maximum cross-sectional dimension of the needle-shaped portions 12 may be, for example, 25 to 1,000 μm. The tip diameter or cross-sectional dimension of tips may be 1 to 100 μm. The height of the needle-shaped portions 12 may be, for example, 50 to 2,000 μm. The needle-shaped portions 12 may be arranged in a plurality of rows in one direction of the base material 11, and each row may be provided with a plurality of needle-shaped portions 12 to form a matrix.

The needle-shaped portions 12 are composed of resin. In the present embodiment, the resin that constitutes the needle-shaped portions 12 is a low-melting-point resin and has a weight-average molecular weight of 25,000 or more, that is, a high-molecular-weight and low-melting-point resin. The low-melting-point resin refers to a thermoplastic resin that is solid at room temperature and has a melting point of 130° C. or lower. As the low-melting-point resin, a material having a melting point of 40° C. to 120° C. may be particularly preferred, and a material having a melting point of 45° C. to 100° C. may be most preferred. Being solid at room temperature allows the shape of the needle-shaped portions 12 to be maintained at room temperature, and when the melting point is 130° C. or lower, there is no need to heat the needle-shaped portions 12 at a high temperature, allowing them to be produced at low cost with good workability. Moreover, even when the resin is bonded to the base material 11 in a molten state or the resin is heated in a state in which the resin and the base material are bonded together, the base material 11 does not soften, deform, or burn, and there is a high degree of freedom in the selection of the base material 11. Furthermore, even when a nonwoven fabric or resin film made of synthetic fibers or the like having a low heat resistance is used as the base material 11, deterioration of the base material 11 due to softening of the synthetic fibers can be prevented.

The weight-average molecular weight of the low-melting-point resin is 25,000 or more, but may be preferably 40,000 to 200,000 and more preferably 60,000 to 150,000. When the weight-average molecular weight is within this range, the needle-shaped portions 12 can maintain n the necessary strength. When the weight-average molecular weight of the low-melting-point resin is 25,000 or more, the water absorbability of the needle-shaped portions 12 is improved. The reason for this is not necessarily clear, but it is presumed that the use of a high-molecular-weight and low-melting-point resin causes the structure of hole portions 13 possessed by the needle-shaped portions 12 to differ from that when using a low-molecular-weight and low-melting-point resin. In the case in which the weight-average molecular weight of the low-melting-point resin is 60,000 or more, the water absorbability of the needle-shaped portions 12 can be further improved when the needle-shaped portions 12 contain a water-insoluble hydrophilic resin, which will be described later.

The tip strength of the needle-shaped portions 12 thus obtained by containing a high-molecular-weight and low-melting-point resin may be usually 100 mN or more, preferably 150 mN or more, and more preferably 200 mN or more. Provided that the tip strength is 100 mN or more, chipping or the like of the needle-shaped portions 12 can be suppressed with a high possibility even when they are pierced into the skin, and the microneedle structure 10 can be used, for example, as a test patch or the like. The tip strength of the needle-shaped portions 12 is a value measured by the procedure described in the examples, which will be described later.

As will be described later, in the case of a structure in which a plurality of hole portions 13 are opened on the side surface of each needle-shaped portion 12, it is possible to increase the rate of absorption or release of fluid from the needle-shaped portions as compared with a structure in which hole portions are opened only at the top portion of each needle-shaped portion, but it is conceivable that the needle-shaped portions 12 may become brittle and the strength tends to be low. Fortunately, however, in the present embodiment, since the needle-shaped portions 12 are configured using a low-melting-point resin having a weight-average molecular weight of 25,000 or more, the strength of the needle-shaped portions 12 can be increased, in particular, the tip strength of the needle-shaped portions 12 can be increased, and it is possible to suppress the breakage of the needle-shaped portions 12, for example, when piercing them into the skin.

The high-molecular-weight and low-melting-point resin that constitutes the needle-shaped portions 12 may be a water-insoluble resin. By being water-insoluble, the resin is not dissolved with fluids containing water, such as body fluids, when applied to a living body, and it is possible to maintain the shape of the microneedle structure 10 for a desired application time. Moreover, as will be described later, the fine hole portions 13 can be readily formed. Examples of water-insoluble resins include: polyolefin-based resins such as polyethylene and α-olefin copolymers; olefin copolymer-based resins such as ethylene-vinyl acetate copolymer resins; polyurethane-based elastomers; and acrylic copolymer-based resins such as ethylene-ethyl acrylate copolymers. From the viewpoint of reducing solubility in water, it is preferred that the water-insoluble low-melting-point resin should not have hydrophilic functional groups such as hydroxyl groups, carboxyl groups, sulfonic acid groups, amine groups, or acetamide groups, except at the ends.

The high-molecular-weight and low-melting-point resin that constitutes the needle-shaped portions 12 may also be a biodegradable resin. Here, the biodegradable resin is a plastic that is completely decomposed into CO₂ and water by microorganisms present in nature after use, and being a biodegradable resin makes it possible to reduce the influence on living organisms. Preferred examples of such biodegradable resins for use include aliphatic polyesters and derivatives thereof, homopolymers of at least one monomer selected from the group consisting of glycolic acid, lactic acid, and caprolactone, and copolymers composed of two or more monomers. In addition, polybutylene succinate (melting point: 84° C. to 115° C.), aliphatic-aromatic copolyester (melting point: 110° C. to 120° C.), etc. can also be used as the low-melting-point biodegradable resin. Specific examples of the polybutylene succinate for use include BioPBS provided by Mitsubishi Chemical Corporation, and specific examples of the aliphatic-aromatic copolyester for use include Ecoflex available from BASF.

The biodegradable resin may be a resin whose monomer acid dissociation constant is 4 or more. When the monomer acid dissociation constant is 4 or more, it is possible to reduce the influence on a living body upon application of the microneedle structure 10 of the present invention to the living body. When the monomer is a cyclic ester, the monomer acid dissociation constant as referred to herein is the acid dissociation constant of the hydroxycarboxylic acid resulting from ring-opening of the cyclic ester. The monomer acid dissociation constant may be preferably 4.0 or larger and further preferably 4.5 or larger. From another aspect, the monomer acid dissociation constant may be preferably 25 or less and further preferably 15 or less. Examples of monomers constituting such a biodegradable resin and having an acid dissociation constant of 4 or more include caprolactone. The constituent units of monomers having an acid dissociation constant of 4 or more from which the low-melting-point biodegradable resin is derived may preferably account for 70 mass % or more, more preferably 80 mass % or more, and further preferably 90 mass % or more in the entire constituent units.

From the viewpoint of efficiently obtaining the effect of being able to process the resin at low temperature, the ratio of the low-melting-point resin to the total mass of the resin components contained in the needle-shaped portions 12 may be preferably 50 mass % or more, more preferably 65 mass % or more, and further preferably 80 mass % or more. The needle-shaped portions 12 may further contain a high-melting-point resin having a melting point higher than 130° C. within a range in which the effect of being able to process the resin at low temperature is not impaired. Examples of high-melting-point resins include biodegradable resins such as polyglycolic acid (melting point: 218° C.), polylactic acid (melting point: 170° C.), and polyhydroxybutyric acid (melting point: 175° C.).

Most preferably, the resin constituting the needle-shaped portions 12 may be a water-insoluble high-molecular-weight and low-melting-point resin that is also biodegradable. Examples thereof include polycaprolactone or a copolymer of caprolactone and another polymer, whose monomer acid dissociation constant is 4 or more.

The needle-shaped portions 12 may preferably contain a water-insoluble hydrophilic resin from the viewpoint of improving the water absorbability of the needle-shaped portions 12. The water-insoluble hydrophilic resin is a high-molecular substance that is insoluble in water and has a hydrophilic functional group. Since the water-insoluble hydrophilic resin is insoluble in water, it is not dissolved with fluids containing water, such as body fluids, when applied to a living body, and it is possible to maintain the shape of the microneedle structure 10 for a desired application time. In addition, by containing a water-insoluble hydrophilic resin, it is possible to easily form fine hole portions 13 in the needle-shaped portions 12 as will be described later. Examples of hydrophilic functional groups include hydroxyl groups, carboxyl groups, sulfonic acid groups, amine groups, and acetamide groups, and hydroxyl groups and carboxyl groups may be preferred. The water-insoluble hydrophilic resin may preferably have a hydrophilic functional group in the main chain or side chain. The carboxyl group may be in the state of a carboxylate under the presence of a counterion such as a metal ion.

As the water-insoluble hydrophilic resin, a resin having both a repeating unit that has a hydrophilic functional group and a repeating unit that does not have a hydrophilic functional group may be used. In this case, however, it is preferred that the mass of the repeating unit having a hydrophilic functional group should account for more than half of the mass of the resin. More preferably, the water-insoluble hydrophilic resin may contain a resin in which all repeating units have hydrophilic functional groups.

The equivalent weight of the hydrophilic functional group in the water-insoluble hydrophilic resin may be, for example, 1,500 or less, preferably 1,100 or less, more preferably 900 or less, and further preferably 500 or less.

Examples of the water-insoluble hydrophilic resin include fully saponified polyvinyl alcohol; and water-insoluble polysaccharides such as cellulose, calcium alginate, chitin, and cross-linked hyaluronic acid. Among these, water-insoluble polysaccharides, which are substances derived from living organisms, may be preferred from the viewpoint of affinity with living organisms, and cellulose may be preferred from the viewpoint of keeping raw material costs low.

The amount of water-insoluble hydrophilic resin contained in the needle-shaped portions 12 may be preferably 4 mass parts or more and 50 mass parts or less, more preferably 5 mass parts or more and 45 mass parts or less, and further preferably 15 mass parts or more and 40 mass parts or less with respect to 100 mass parts of the high-molecular-weight and low-melting-point resin from the viewpoints of further improving the water absorbability of the needle-shaped portions 12 and facilitating the preparation of compositions for forming the needle-shaped portions 12. The water-insoluble hydrophilic resin is usually not compatible with the low-melting-point resin and exists in the needle-shaped portions 12 in a state separated from the low-melting-point resin.

The needle-shaped portions 12 may contain a filler. By containing a filler in the needle-shaped portions 12, it is possible to further improve the mechanical strength of the needle-shaped portions 12. The filler may be preferably contained so that it is in a dispersed state in the resin of the needle-shaped portions 12.

The filler may be preferably composed of a resin, or one selected from the group consisting of natural organic polymers or modified products thereof and biodegradable resins. The filler for use composed of a resin may be one that contains an inorganic component, etc., such as an organic/inorganic hybrid filler in which an inorganic material is attached to the surfaces of resin particles, but considering the influence on a living body, the filler may be preferably composed only of a resin and an organic component, and more preferably composed only of a resin. Examples of natural organic polymers include polysaccharide such as cellulose, and examples of fillers composed of natural organic polymers or modified products thereof include cellulose fibers and cellulose acetate true spherical particles. The above-described water-insoluble polysaccharide may be contained as polysaccharide in the needle-shaped portions 12 in the form of particles so as to function as a filler.

The biodegradable resins described above can be used, but when a biodegradable resin is used as the high-molecular-weight and low-melting-point resin, it is preferred to use a biodegradable resin different from this biodegradable resin, and from the viewpoint of further improving the mechanical strength of the filler as described below, a biodegradable resin whose melting point exceeds 130° C. or a biodegradable resin with no melting point may be preferred. Examples of such biodegradable resins include polylactic acid (melting point: 170° C.), polyglycolic acid (melting point: 218° C.), polyhydroxybutyric acid (melting point: 175° C.), and cellulose acetate diacetate (melting point: 230° C. to 300° C.). Biodegradable resins such as cellulose butyrate diacetate also fall under modified products of natural organic polymers.

The filler may be preferably composed of a resin whose melting point exceeds 130° C. or a resin having no melting point. Resins whose melting point exceeds 130° C. are less likely to soften at temperatures near an ordinary temperature at which the microneedle structure 10 is used. Therefore, when the filler is composed of a resin whose melting point exceeds 130° C., it is easy to obtain sufficient strength of the microneedle structure 10. In addition, when the filler is composed of a resin whose melting point exceeds 130° C., addition of such a resin in the form of a filler may be preferred because when such a resin that is difficult to melt is mixed with a low-melting-point resin in a state in which the filler is dispersed in the composition, production is possible through low-temperature kneading without melting of the filler. Other than the above-described biodegradable resins, examples of a resin whose melting point exceeds 130° C. and a resin having no melting point include polypropylene (melting point: 155° C.), polybutylene terephthalate (223° C.), polyethylene terephthalate (melting point: 260° C.), polytetrafluoroethylene (melting point: 327° C.), melamine resin (melting point: none), and unmodified cellulose (melting point: none).

The filler may be preferably composed of a resin whose glass-transition temperature is 80° C. or lower. Provided that the filler is composed of a resin whose glass-transition temperature is 80° C. or lower, even when melting is performed at a low temperature for the resin constituting the needle-shaped portions 12, the filler is readily softened during the melting and is more compatible with the resin constituting the needle-shaped portions 12. This can readily improve the strength of the needle-shaped portions 12 to be produced. When the resin contained in the filler is crosslinked, the glass-transition temperature of the polymer being 80° C. or lower is determined before crosslinking. Examples of resins whose glass-transition temperature (Tg) is 80° C. or lower include polypropylene (Tg: 0° C.), polybutylene terephthalate (Tg: 50° C.), polyethylene terephthalate (Tg: 69° C.), polymethyl methacrylate (Tg: 60° C.), polylactic acid (Tg: 60° C.), polyglycolic acid (Tg: 40° C.), and polyhydroxybutyric acid (Ig: 15° C.), among which, as described above, a biodegradable resin may be preferred, and polylactic acid, polyglycolic acid, polyhydroxybutyric acid, or a copolymer of monomers of these polymers may be preferred. From the viewpoint of further improving the mechanical strength of the needle-shaped portions 12, the filler may also be preferably composed of a resin whose glass-transition temperature is −10° C. or higher. The filler may more preferably composed of a resin whose glass-transition temperature is 10° C. to 80° C., and further preferably composed of a resin whose glass-transition temperature is 30° C. to 75° C.

The filler may be preferably contained in an amount of 3 to 50 mass %, more preferably 5 to 43 mass %, and further preferably 10 to 35 mass % with respect to the total mass of the needle-shaped portions 12. When it is 50 mass % or less, the shape of the needle-shaped portions 12 may readily be maintained, and the workability during production can be improved. When it is 3 mass % or more, it may be easier to increase the strength. When the filler is contained in this content range, it may be easy to increase the strength of the needle-shaped portions 12 by the filler while maintaining the liquid permeability by forming the needle-shaped portions 12 with a desired porosity. Two or more types of the above-described fillers may be contained. Also in this case, it may be preferred to contain the filler so that the total amount of the filler is within the above content range with respect to the resin constituting the needle-shaped portions 12. When the filler is a water-insoluble polysaccharide, the content of the filler, which is a water-insoluble polysaccharide, may be preferably 4 mass parts or more and 50 mass parts or less, more preferably 5 mass parts or more and 45 mass parts or less, and further preferably 15 mass parts or more and 40 mass parts or less with respect to 100 mass parts of the high-molecular-weight and low-melting-point resin.

The shape of the filler may be a plate-like (flake-like) shape, a fibrous shape, a spherical shape, an indefinite shape, or the like, but may be preferably a fibrous shape. When the shape of the filler is a fibrous shape, it is more compatible with the resin constituting the needle-shaped portions 12 in a molten state, and the strength of the needle-shaped portions 12 obtained is more readily improved, which may be preferred. Examples of fillers having a fibrous shape include metal fiber filler, carbon fiber, carbon nanofiber, and cellulose fiber. When the filler is in a shape other than a fibrous shape, for example, when the filler is in a spherical shape or an indefinite shape, the filler may be composed of a resin whose glass-transition temperature is 80° C. or lower, as described above, thereby to allow the filler to be more compatible with the low-melting resin. The particle diameter of the filler may be 0.3 to 150 μm, preferably 0.5 to 125 μm, and more preferably 1 to 100 μm. When the particle diameter of the filler is 0.3 to 150 μm, the filler may be more readily dispersed in a composition containing a low-melting resin, and the strength of the microneedle structure 10 obtained can be further improved. The particle diameter of the filler is a seven-point average of the values obtained through observing the filler in the microneedle structure 10 with a scanning electron microscope (SEM) and measuring the lengths of the longest portions of the particles. When the filler is in a fibrous shape, the particle diameter refers to the fiber length.

The needle-shaped portions 12 are each formed with the hole portions 13 as flow channels that allow liquids to flow inside. One or more hole portions 13 may be formed in one needle-shaped portion 12 and opened at the surface of the needle-shaped portion 12. The hole portions 13 may be formed in any manner, for example, a single communicating hole may be mechanically provided, but it may be preferred to form the needle-shaped portions 12 with porous structures as in the present embodiment. When each needle-shaped portion 12 is formed so that at least a part thereof has a porous structure, body fluids or drug solutions can pass through the hole portions 13 of the porous structure, so this may be preferred because nano-order flow paths are not necessary to be mechanically formed. Moreover, body fluids or drug solutions can flow through all the flow paths of the portion formed with the porous structure in each needle-shaped portion 12, and the amount of flow can therefore be increased as compared with when a simple single communicating hole is formed. When the needle-shaped portions 12 are formed with porous structures, the surface area of the hole portions 13 with which fluids containing water, such as body fluids or drug solutions, come into contact inside the needle-shaped portions 12 is large. Thus, when the needle-shaped portions 12 contain a water-insoluble hydrophilic resin, the hydrophilicity of the surfaces of the hole portions 13 can be increased thereby to readily obtain the effect of improving the water absorbability of the needle-shaped portions 12. Furthermore, when each needle-shaped portion 12 is thus formed so that at least a part thereof has a porous structure, if the porous structure is not covered partially or entirely on the side surface of a needle-shaped portion 12, the hole portions 13 are also opened on the side surface of that needle-shaped portion 12. In this case, the amount of flow of the liquid can be increased as compared with when only the tip portion of a needle-shaped portion 12 is opened.

In such a case, it is conceivable that the needle-shaped portions 12 may become brittle. Fortunately, however, in the present embodiment, the needle-shaped portions 12 are formed using a low-melting-point resin having a weight-average molecular weight of 25,000 or more and a melting point of 130° C. or lower, and it is therefore possible to form the needle-shaped portions 12 which are not brittle and have high strength.

The method of forming the porous structures will be described in detail later, but a method of forming the porous structures simultaneously with the formation of the needle-shaped portions 12, or a method of forming projecting portions 32 formed with no porous structures (not illustrated in FIG. 1, described later) and then forming porous structures in the projecting portions 32, may be preferred from the viewpoint of obtaining the hole portions 13 with a continuous structure. In the latter case, for example, the needle-shaped portions 12 of porous structures may be obtained through mixing two or more different materials to form the projecting portions 32 and then removing at least one material to form the hole portions 13. When the needle-shaped portions 12 contain a filler, according to such a method of forming the porous structures, the filler is contained in a dispersed state in the resin of the needle-shaped portions 12. In the present embodiment, the needle-shaped portions 12 are composed of high-molecular-weight polycaprolactone and, as will be described later, are formed through creating the projecting portions 32 composed of polycaprolactone, which is a water-insoluble resin, and a water-soluble material, removing the water-soluble material, which is soluble in water, in a removal step to form the hole portions 13, and leaving the water-insoluble resin, which is insoluble in water, to form the porous needle-shaped portions 12.

Thus, the hole portions 13 are voids formed by removing the water-soluble material from the projecting portions 32 composed of the water-insoluble high-molecular-weight and low-melting-point resin and the water-soluble material, and the body fluid or drug solution passes through the hole portions 13 which serve as flow channels. As illustrated in the cross sections of the needle-shaped portions 12, the hole portions 13 are formed by removing the water-soluble material to form a plurality of voids that communicate with each other. Some of the hole portions 13 may communicate from the surfaces of the needle-shaped portions 12 to one surface of the base material 11 to form the flow channels. The size of openings of the hole portions 13 is determined by the application such as a test patch using the microneedle structure 10, but from the viewpoint of facilitating the passage of liquids, the size of the openings may be preferably 0.1 to 50.0 μm, more preferably 0.5 to 25.0 μm, and further preferably 1.0 to 10.0 μm. In order to obtain such an opening diameter, the water-soluble material and its content may be appropriately selected in the production steps.

In the present embodiment, the needle-shaped portions 12 are formed by removing the water-soluble material from the projecting portions 32 composed of the water-insoluble high-molecular-weight and low-melting-point resin and the water-soluble material, but the method is not limited to this. It may also be possible to form the needle-shaped portions 12 using a porous high-molecular-weight and low-melting-point resin. The porous structures may also be formed simultaneously with the formation of the needle-shaped portions 12 using a foaming material or the like, or the porous structures may be formed by sintering a particulate composition containing a low-melting-point resin.

The needle-shaped portions 12 may be provided with a base portion 14 that is provided between the needle-shaped portions 12 and one surface side of the base material 11 over at least a region where the needle-shaped portions 12 are formed. In the present embodiment, the base portion 14 is provided in a layered form over the entire one surface of the base material 11. The base portion 14 serves as a base for each needle-shaped portion 12 and has hole portions 13 similarly to each needle-shaped portion 12. The base portion 14 may be formed to have a thickness, for example, of 0.1 to 500 μm. With such a thickness, the strength of the base material 11 is increased, and preferred adhesive properties are obtained between the needle-shaped portions 12, the base portion 14, and the base material 11.

Like the needle-shaped portions 12, the base portion 14 preferably has a porous structure, and it may be more preferred to use the same resin so as to constitute the same porous structure as that of the needle-shaped portions 12. When a porous structure is used for the base portion 14, there is no need to mechanically form hole portions 13 because flow channels through which liquids flow are formed inside the porous structure, and liquids from the needle-shaped portions 12 can pass through the hole portions 13 of the base portion 14 to fill the through-holes 15, which may be preferred. In the present embodiment, the base portion 14 is composed of the same high-molecular-weight and low-melting-point resin as that of the needle-shaped portions 12 and is formed by the same steps; therefore, not only can the base portion 14 be easily created, but also better adhesion can be achieved between the needle-shaped portions 12 and the base material 11 via the base portion 14, which may be preferred. Furthermore, in the present embodiment, since the base portion 14 is provided over the entire one surface of the base material 11, the base portion 14 is present in a state of being attached to the base material 11 even in the portion of the base material 11, which is not formed with the needle-shaped portions 12, and the strength of the microneedle structure 10 is further improved as a whole.

When the needle-shaped portions 12 have a porous structure and are provided with the base portion 14, a water absorption ratio of the needle-shaped portions 12 measured by a testing method below in a state in which the needle-shaped portions 12 are composed only of the porous structure may be preferably 8.5% or more.

### (Testing Method)

The needle-shaped portions are immersed in 10 ml of purified water under an environment of 25° C. The needle-shaped portions in an immersed state are placed under a reduced pressure environment of 0.09 MPa for 1 hour to allow water to penetrate into the interior of the porous structure. Subsequently, the needle-shaped portions are taken out from generation, and water droplets attached to the surface are removed. Water droplets on the surfaces of the needle-shaped portions on the side formed with needles are removed by blowing them off with an air blow gun, and water droplets on the surfaces of the base portion side of the needle-shaped portions are removed through placing the base portion on a glass plate and allowing the needle-shaped portions' own weight to push the water droplets around the base portion, and after statically placing it for 5 seconds, the needle-shaped portions are picked up from the glass plate. Then, weight measurement is performed for a sample after water absorption. The water absorption ratio (ratio of absorbed water to the sample's own weight) is obtained using an equation below.

$$Water absorption ratio ( \% ) = ( weight of sample after water absorption - weight of sample before water absorption ) \div weight of sample before water absorption \times 100$$

Measurement of the water absorption ratio can be specifically performed by the method described in the testing example, which will be described later. When the microneedle structure 10 includes the base material 11 described below, the base material 11 is removed to leave the needle-shaped portions composed only of a porous structure, and the water absorption ratio can be measured in the same manner. Such a water absorption ratio may be more preferably 13% or more, further preferably 20% or more, and even further preferably 28% or more. The upper limit of the water absorption ratio is not particularly limited, but it may be usually about 50% or less.

### (2) Base Material

The needle-shaped portions 12 are each formed with the hole portions 13 as flow channels that allow liquids to flow inside, which may reduce the strength of the needle-shaped portions 12 compared to needle-shaped portions in which the hole portions 13 are not formed. When the needle-shaped portions 12 are formed with a porous structure, the strength of the needle-shaped portions 12 tends to be further reduced. Accordingly, in the present embodiment, the microneedle structure 10 includes the base material 11, which is provided with the needle-shaped portions 12, on one surface side in order to support the needle-shaped portions 12 from the base side of the needle-shaped portions 12 and improve the strength of the microneedle structure 10.

The base material 11 may be preferably configured such that a liquid can pass through the base material 11 in its thickness direction. The feature that a liquid can pass through the base material 11 in its thickness direction may refer to a feature that the base material 11 itself is composed of a material that is permeable to liquids or a feature that the base material 11 is configured such that a liquid can pass through the base material 11 in its thickness direction via the through-holes 15 formed in the base material 11, while being composed of a material that is impermeable to liquids.

An example of the base material 11 composed of a liquid-permeable material may be a porous base material in which a plurality of voids communicate with each other to form fine base material hole portions penetrating from one surface (surface on which the needle-shaped portions 12 are provided) to its back surface (surface opposite to the surface on which the needle-shaped portions 12 are provided). When a low-melting-point resin is used as the resin forming the needle-shaped portions 12, the composition containing the low-melting-point resin can be processed at a low temperature, so that the base material 11 can be prevented from being exposed to high temperatures. Thus, various base materials can be selected as the base material 11 depending on the application. The base material 11 composed of such a liquid-permeable material may be in the form of a plate, but may be preferably in the form of a sheet that has high followability to the skin. The base material 11 may be preferably composed of a fibrous material that is easy to handle. Here, the fibrous material in the present invention means fibers such as natural fibers and chemical fibers. Examples of base materials composed of fibrous materials include nonwoven fabrics, woven fabrics, knitted fabrics, and paper composed of these fibers.

When the base material 11 can pass a liquid through the base material 11 in its thickness direction via the through-holes 15, while being composed of a material that is impermeable to liquids, liquid absorption of the base material 11 can be suppressed, so the liquid can only pass through the through-holes 15 in the base material 11. Therefore, the body fluid obtained from the needle-shaped portions 12 or the drug solution transported to the needle-shaped portions 12 does not seep into the base material 11, so that the entire amount can be transported via the through-holes 15. Through this configuration, when the microneedle structure 10 is used as a test patch, a rapid analysis is possible because the body fluid can pass through the base material 11 in a moment, while also when the microneedle structure 10 is used as a drug administration patch, the drug solution does not seep and the total amount of the drug solution can be quickly supplied to the skin. When the base material 11 has the through-holes 15, the microneedle structure 10 may be configured such that the through-holes are filled with an absorbent material capable of absorbing liquid, as described in International Publication WO2023/042525, and the absorbent material may be a porous material. With such a configuration of the microneedle structure 10, it is possible to speed up the flow of liquids such as body fluids between an analysis sheet 17 described later provided on the back surface side of the base material 11 or the drug storage portion and the living body.

Examples of such liquid-impermeable materials include resin films, metal-containing sheets, and glass films. Examples of metal-containing sheets include metal foil. Among resin films, those with low water resistance may be formed with a metal layer by vapor deposition or the like to improve water resistance, and used as metal-containing sheets. Materials that do not have liquid-impermeability, such as nonwoven fabric or paper, may also be provided as laminated resin films that are configured to be impermeable to liquids as a whole by laminating a water-insoluble resin thereon.

In the present embodiment, the base material 11 is composed of a liquid-impermeable resin film. Examples of resins that can be used for such resin films include resins with relatively low heat resistance selected from the group consisting of polybutylene terephthalate, polyethylene terephthalate, polyethylene, polypropylene, ethylene-vinyl acetate copolymer, vinyl chloride, acrylic resin, polyurethane, and polylactic acid, as well as heat-resistant resins such as polyimide, polyamideimide, and polyethersulfone. In the present embodiment, when a low-melting-point resin is used as the resin forming the needle-shaped portions 12 and the composition containing the low-melting-point resin can be processed at low temperatures, the base material 11 can be prevented from being exposed to high temperatures. Thus, even with a resin film using a resin having low heat resistance, problems such as deformation of the base material are less likely to occur.

The base material 11 may be a single layer or may have a configuration in which multiple layers are laminated. The base material 11 may be a laminate of a porous base material 11 such as a nonwoven fabric and a liquid-impermeable base material 11 formed with through-holes. The resin film may also be a composite film obtained by impregnating a nonwoven fabric or cloth with a resin. The thickness of the base material 11 may be preferably 3 to 200 μm, more preferably 10 to 140 μm, and further preferably 30 to 115 μm. When the thickness is 3 μm or more, it is easy to maintain the strength as the base material 11, and when the thickness is 200 μm or less, the followability to the skin is improved and the liquid transport time can be shortened.

The base material 11 may be provided with an adhesive layer 16 on one surface side on which the needle-shaped portions 12 are formed. This can improve the adhesiveness between the needle-shaped portions 12 and base portion 14 and the base material 11. As such an adhesive layer, a pressure sensitive adhesive may be preferred, and examples thereof include an acrylic-based pressure sensitive adhesive, a silicone-based pressure sensitive adhesive, and a rubber-based pressure sensitive adhesive, among which the acrylic-based pressure sensitive adhesive can be more preferably used. By providing the adhesive layer 16 on the base material 11, the microneedle structure 10 can be easily obtained in a method for producing a microneedle structure, which will be described later, through preliminarily making a solid composition 31 adhere to the base material 11, putting the base material 11 and the solid composition 31 into a mold, and heating and pressing them in a heating and pressurizing step. When the adhesive layer 16 is provided on the base material 11, there is a concern that a gap may be generated between the base material 11 and the needle-shaped portions 12, causing liquid to leak out, or that the adhesive layer may prevent the passage of liquids between the base material 11 and the needle-shaped portions 12. For this reason, it may be preferred to provide the adhesive layer 16 so that it surrounds a region of the base material 11 through which the liquids should pass, while providing a central region in which the adhesive layer 16 is not formed. Although such an effect cannot be obtained, a first primer layer (not illustrated) may be provided as substitute for the adhesive layer 16, for example, for the purpose of improving the adhesiveness between the needle-shaped portions 12 and the base material 11. Even when the base material 11 has the adhesive layer 16, the first primer layer as an intermediate layer may be provided between the base material 11 and the adhesive layer 16. Examples of the primer layer include an acrylic-based primer layer and a polyester-based primer layer.

As the acrylic-based pressure sensitive adhesive, one containing an acrylic polymer obtained by polymerizing a monomer whose main component is an alkyl acrylate can be used. The acrylic-based polymer may be a copolymer of an alkyl acrylate and another monomer. Examples of the other monomer include acrylic esters other than alkyl acrylates, such as acrylic esters having a hydroxyl group, acrylic esters having a carboxyl group, and acrylic esters having an ether group and monomers other than acrylic esters, such as vinyl acetate and styrene.

The acrylic-based polymer may be crosslinked by a reaction between a functional group derived from the above-described acrylic ester having a hydroxyl group, acrylic ester having a carboxyl group, etc., and a crosslinker.

The acrylic-based pressure sensitive adhesive may contain a tackifier, a plasticizer, an antistatic, a filler, a curable component, etc. in addition to the above-described components.

As a coating liquid for obtaining the acrylic-based pressure sensitive adhesive, any of a solvent-based one and an emulsion-based one can be used.

The shape of the through-holes 15 formed in the base material 11 is not particularly limited, but from the viewpoint of ensuring a sufficient amount of liquid flow while causing capillary action, a structure provided with a plurality of through-holes having a small diameter may be preferred. The diameter of the through-holes 15 may be, for example, 2 mm or less, preferably 0.05 to 1 mm, and more preferably 0.1 to 0.8 mm. The method of forming the through-holes 15 is not particularly limited, and the through-holes 15 may be formed, for example, by laser perforation. In the present embodiment, when transporting a liquid from the needle-shaped portions 12, the liquid does not seep into the base material 11 because the base material 11 has liquid impermeability, and the transportation distance is short because the liquid flows through the through-holes 15 in the thickness direction of the base material 11; therefore, when configured as a detection patch, it can perform the detection at a high analysis speed, while when configured as a drug administration patch, it can administer the drug solution early.

The sum of the areas of the through-holes 15 (total area) may be preferably 0.05% to 15%, more preferably 0.75% to 10%, and further preferably 1% to 5% in total with respect to the area of the region on the base material 11 in which the through-holes 15 are provided. When the total area of the through-holes 15 is 15% or less with respect to the area of the above region, the rigidity of the base material 11 can be readily ensured. On the other hand, when the total area of the through-holes 15 is 0.05% or more with respect to the area of the above region, the body fluid can be more efficiently acquired via the base material 11.

When the base portion 14 is formed in the microneedle structure 10, the base portion 14 is directly bonded to one surface of the base material 11, and the base portion 14 is integrally formed with the needle-shaped portions 12, so that the needle-shaped portions 12 are provided on the base material 11 without the use of adhesive or the like, and the hole portions 13 well communicate one another, allowing liquids to pass through easily. The microneedle structure 10 having such a configuration can be obtained, even when the adhesive layer 16 is not provided on the base material 11, through bonding the solid composition 31 to the base material 11 by heating in the formation step in the method for producing a microneedle structure, which will be described below, or through a similar bonding method using heating. In the present embodiment, the base portion 14 is provided over the entire surface of the base material 11, but the present invention is not limited to this. The base portion 14 may be preferably formed at least in the region in which the needle-shaped portions 12 are formed. Even when the base portion 14 is directly bonded to one surface of the base material 11, as described above, the base material 11 may be provided with the first primer layer as substitute for the adhesive layer 16, and the base portion 14 may be bonded to the base material 11 via the first primer layer, or via a layer other than the adhesive layer 16 and the first primer layer.

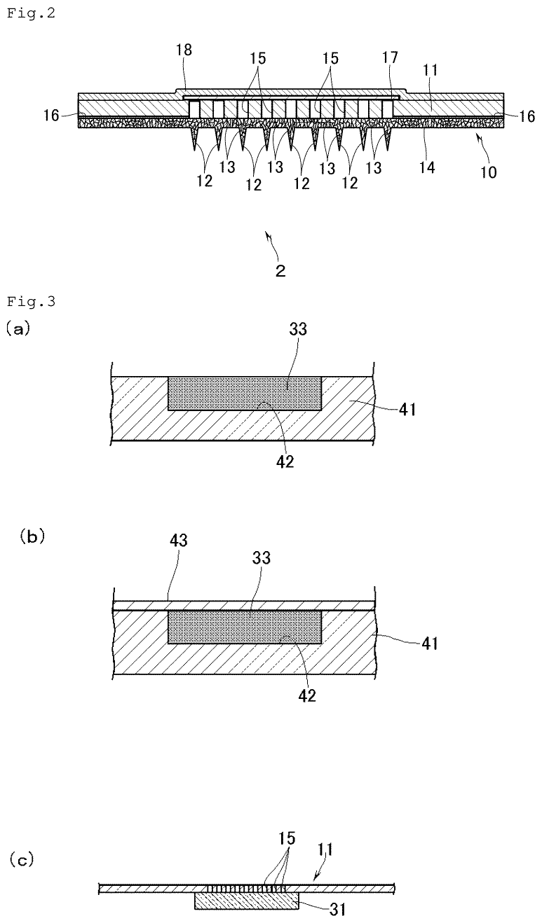

The microneedle structure 10 thus formed can be used as a test patch or a drug administration patch. For example, as illustrated in FIG. 2, in a test patch 2, an analysis sheet 17 is disposed in a region position of the base material 11 of the obtained microneedle structure 10 in which the through-holes 15 are formed, facing the needle-shaped portions 12, and a tape 18 is laminated to cover the analysis sheet 17. In the case of a drug administration patch, it may be configured such that a drug administration member is disposed as substitute for the analysis sheet 17 in a position covering a region of the base material 11 of the obtained microneedle structure 10 in which the through-holes 15 are formed, facing the needle-shaped portions 12, and the tape 18 is laminated to cover the drug administration member. In such a test patch or drug administration patch, since the strength of the needle-shaped portions 12 is high, it is possible to pierce the needle-shaped portions 12 into the skin without the defects, and the constituent material of the needle-shaped portions 12 can be prevented from remaining in the body, which may be preferred. The tape 18 for fixing the analysis sheet 17 or drug administration member to the base material 11 may be a pressure sensitive adhesive tape provided with a pressure sensitive adhesive layer.

### <Method for Producing Microneedle Structure>

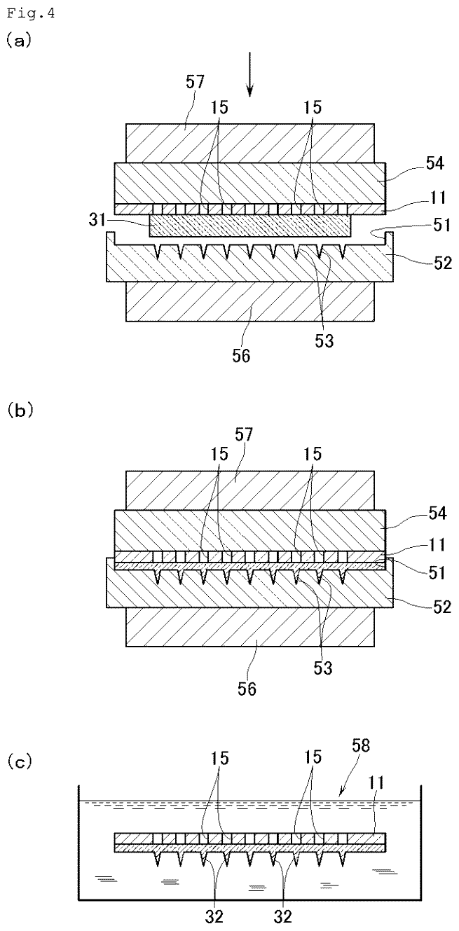

FIGS. 3 and 4 illustrate a method for producing the microneedle patch 1 and test patch 2 according to an embodiment of the present invention. The method of the present embodiment includes melting a water-insoluble high-molecular-weight and low-melting-point resin and a water-soluble material for forming the hole portions 13 to fill a mold with them (filling step), solidifying the filled mixture to obtain a solid composition 31, bonding the solid composition 31 obtained by solidifying the filled mixture to the base material 11 (bonding step), then heating and pressurizing the solid composition 31 to form the projecting portions 32 (formation step), and thereafter removing the water-soluble material from the projecting portions 32 (removal step) to form the projecting portions 32 into the needle-shaped portions 12. This will be described in detail below.

### (Filling Step)

Creation of the base material 11 and solid composition 31 will first be described. First, a composition containing a water-insoluble high-molecular-weight and low-melting-point resin, a water-soluble material, and optional components (e.g., a water-insoluble hydrophilic resin and a filler) is heated to melt and mixed to prepare a mixture 33.

In the present embodiment, the shape of the high-molecular-weight and low-melting-point resin is not particularly limited, and a commonly used pellet-shaped resin can be used.

In preparation of the mixture 33, it may be preferred to heat the resin at 40° C. or higher and 180° C. or lower, more preferably at 55° C. to 180° C., and further preferably at 70° C. to 170° C. so that the viscosity of the resin can be reduced when it is melted. In the present embodiment, the heating temperature can be set relatively low also in preparation of the mixture 33 because a water-insoluble high-molecular-weight and low-melting-point resin is used as the resin constituting the needle-shaped portions 12. Accordingly, even if the base material 11 is heated together with the solid composition 31 in the subsequent formation step to form the projecting portions 32, it is heated at a low temperature; therefore, the base material 11 is low in cost and has good workability, and the base material 11 does not soften, deform, or burn, so that the degree of freedom in selecting the base material 11 is high. When using a pellet-shaped high-molecular-weight and low-melting-point resin, the high-molecular-weight and low-melting-point resin and the water-soluble material can be sufficiently kneaded using a kneader. The mixture 33 may be preferably in a molten state. When it is important to perform heating at a lower temperature, the mixture 33 may be softened to an extent that allows it to be bonded to the base material 11, but in consideration of reducing the production time, etc., it may be preferred to heat the mixture 33 at a temperature equal to or higher than the melting point of the low-melting-point resin, as described above, at which the water-insoluble material begins to melt.

As the water-soluble material, a water-soluble material having at least a melting point higher than an ordinary temperature may be preferred. The water-soluble material may be organic or inorganic, and examples thereof include sodium chloride, potassium chloride, salt cake, sodium carbonate, potassium nitrate, alum, sugar, and water-soluble resin. The water-soluble resin may be preferably a water-soluble thermoplastic resin, and may preferably have a melting point higher than an ordinary temperature. Examples of the water-soluble thermoplastic resin include hydroxypropylcellulose and polyvinylpyrrolidone in addition to biodegradable resins, which will be described below. The water-soluble thermoplastic resin may be more preferably a biodegradable resin in consideration of the influence on the human body. Such biodegradable resins include at least one selected from the group consisting of polyalkylene glycols such as polyethylene glycol and polypropylene glycol, polyvinyl alcohol, collagen, and a mixture thereof, and polyalkylene glycol may be particularly preferred. The molecular weight of polyalkylene glycol is, for example, preferably 200 to 4,000,000, more preferably 600 to 500,000, and particularly preferably 1,000 to 100,000. It may be preferred to use polyethylene glycol among polyalkylene glycols.

In order that both the high-molecular-weight and low-melting-point resin and the water-soluble material can be readily melted at the same heating temperature when preparing the mixture 33, the difference between the melting point of the high-molecular-weight and low-melting-point resin and the melting point of the water-soluble material may be preferably 40° C. or less and more preferably 30° C. or less.

The water-insoluble material and the water-soluble material may be preferably mixed at a mass ratio of 9:1 to 1:9, more preferably 8.5:1.5 to 3:7, and particularly preferably 8:2 to 5:5. When the mixture 33 is configured in this ratio, the needle-shaped portions 12 having a desired porosity can be formed, and the needle-shaped portions 12 can readily achieve both the liquid permeability and the strength.

As illustrated in FIG. 3(a), the mixture 33 is injected into a recessed portion for solid composition 42 formed in a mold for solid composition 41. The recessed portion for solid composition 42 may be formed with a shape and a capacity that allow a desired amount of the mixture 33 to be stored.

The material of the mold for solid composition 41 is also not particularly limited, but it may be preferably formed, for example, of a silicone compound or the like, which facilitates the creation of an accurate mold and allows the solid composition 31 obtained by solidification to be readily released. In the present embodiment, the mold for solid composition 41 is composed of polydimethylsiloxane.

### (Bonding Step)

As illustrated in FIG. 3(b), in a state in which the mixture 33 is stored in the recessed portion for solid composition 42, a sheet for solid composition 43 composed, for example, of polydimethylsiloxane (PDMS) is placed as a lid on the upper surface of the recessed portion for solid composition 42 to flatten the surface of the solid composition 31 obtained. By holding the entire mold for solid composition 41 at −10° C. to 3° C. for 1 to 60 minutes, the molten mixture 33 solidifies and becomes solid, so it is released with the sheet for solid composition 43 from the mold for solid composition 41, and the sheet for solid composition 43 is then removed. Through this operation, the solid composition 31 is obtained.

The base material 11 is prepared. In the present embodiment, the base material 11 has the adhesive layer 16. The adhesive layer 16 may be formed by coating or application, but in the present embodiment, a pressure sensitive adhesive tape having the adhesive layer 16 in a predetermined region is used as the base material 11. Then, through-holes 15 are formed in the base material 11. The method of forming the through-holes 15 is not particularly limited, and the through-holes 15 may be formed, for example, by laser perforation.

Then, as illustrated in FIG. 3(c), the solid composition 31 is attached to the adhesive layer 16 of the base material 11 to integrate the base material 11 and the solid composition 31. Thus, by having the first adhesive layer 16, the microneedle structure 10 can be easily obtained through preliminarily making the solid composition 31 adhere to the base material 11 and placing the base material 11 and the solid composition 31 in the heating and pressurizing step, which will be described later. Moreover, the base material 11 and the solid composition 31 are integrated, so the handling such as transportation will be easier.

### (Formation Step)

Then, as illustrated in FIG. 4(a), the solid composition 31 with the base material 11 is placed in a recessed portion 51 of a mold 52. Projection forming recessed portions 53 are also provided around the center of the bottom surface of the recessed portion 51. The solid composition 31 is placed on the bottom surface of the recessed portion 51, that is, on the projection forming recessed portions 53. The projection forming recessed portions 53 are for forming the needle-shaped portions 12 and are formed in a shape and size corresponding to the needle-shaped portions 12. Then, a lid 54 of the mold 52 is installed on the other surface side (back surface side) of the base material 11. This lid 54 is also composed, for example, of polydimethylsiloxane.

Then, the heating step illustrated in FIG. 4(b) is performed. The heating step is for forming the projecting portions 32 having a desired form, etc., and the heating and pressurization may be performed once, but as in the present embodiment, in order to sufficiently fill the recessed portion 51 of the mold 52 with the solid composition 31, the formation step preferably includes a preliminary step for starting to melt the solid composition 31 provided with the base material 11 and a main step for sufficiently filling the recessed portion 51, etc. with the molten solid composition 31.

First, in the preliminary step and the main step, as illustrated in FIG. 4(b), the base material 11 and the solid composition 31 are interposed between the mold 52 and the lid 54 in a state in which the solid composition 31 is placed on the recessed portion 51. Then, in this state, the mold 52 and the lid 54 are placed on a lower stage 56, and an upper stage 57 is installed on the mold 52 and the lid 54.

As for the heating conditions in the preliminary step and the main step, the heating may be performed at 40° C. or higher and 180° C. or lower at which the influence on the base material 11 is small, preferably at 55° C. to 180° C., and further preferably at 70° C. to 170° C. In the present embodiment, the heating is performed at a temperature at which the solid composition 31 can melt. In order to heat the solid composition 31, at least one of the lower stage 37 and the upper stage 57 may be heated or both may also be heated, but it may be preferred to heat both. In order to quickly fill the recessed portion 51 and the like with the solid composition 31 containing a high-molecular-weight and low-melting-point resin, it may be preferred to set the lower stage 56 at a high temperature, and for example, the lower stage may be set at a temperature in a range of 120° C. to 180° C. The temperature of the upper stage 57 may be preferably set at a temperature in a range of 70° C. to 110° C. from the viewpoint of suppressing deformation or the like of the base material due to heat while obtaining the effect of improving the adhesiveness between the needle-shaped portions 12 or base portion 14 and the base material 11, as described later. In the main step, the heating may be maintained after the preliminary step, and the temperature may be changed as appropriate.

In this state, the mold 52 is pressed (pressurized) between the upper stage 57 and the lower stage 56. The pressure in this preliminary step is preferably 0.1 to 5.0 range allows the solid MPa. The pressure within this composition 31 to be melted in a short time and allows the molten solid composition 31 to quickly fill the recessed portion 51, etc. Then, the retention for 10 seconds to 10 minutes leads to a state in which the solid composition 31 is melted. The pressurization conditions may be changed between the preliminary step and the main step. For example, in the main step, pressurization can be performed at a higher pressure or for a longer time than in the preliminary step.

By performing the preliminary step and the main step as in the present embodiment, the solid composition 31 is sufficiently melted and fills the recessed portion 51 and the projection forming recessed portions 53. When the obtained needle-shaped portions 12 or base portion 14 has a porous structure, the bonding area of the needle-shaped portions 12 or the base portion 14 with respect to the base material 11 will be small, which may be disadvantageous for the bonding properties therebetween. Fortunately, however, the base material 11 and the solid composition 31 are subjected to the heating in the formation step in a state of being bonded to each other, and it can thereby be possible to improve the bonding properties between the needle-shaped portions 12 or the base portion 14 and the base material 11.

After that, the mold 52 is released from the lower stage 37, and the molten solid composition 31 is retained at −10° C. to 3° C. for 1 to 60 minutes to be refrigerated and solidified (refrigeration/solidification step). This allows the projecting portions 32, etc. to be formed, which have high transferability with a shape corresponding to the projection forming recessed portions 53.

### (Removal Step)

After the bonding step is completed, a product in which the solidified projecting portions 32 and the base material 11 are bonded to each other is released from the mold 52 and statically placed in a liquid, and a removal step is performed to remove the water-soluble material to form the needle-shaped portions 12.

The cleaning liquid in this removal step contains water, and in the present embodiment, as illustrated in FIG. 4(c), the removal step is performed by statically placing in a cleaning liquid 58 the product in which the projecting portions 32 and the base material 11 are bonded to each other. By statically placing it in the cleaning liquid containing water, portions exposed to outside or portions communicating with the portions exposed to outside in the water-soluble material contained in the projecting portions 32, etc. dissolve and flow into the water and are removed. The cleaning liquid is sufficient if it contains water, and may be, for example, a mixed solvent of water and alcohol, or the like. Through this removal, the hole portions 13 are formed in the projecting portions 32, etc., and the needle-shaped portions 12 composed of the remaining high-molecular-weight and low-melting-point resin are formed. Also in the molten solid composition 31 attached to one surface of the base material 11 by being filled in the recessed portion 51, the water-soluble material is removed so that the base portion 14 is also formed with the same porous structure as in the needle-shaped portions 12. This allows the microneedle structure 10 of the present embodiment to be obtained.

### (Method for Producing Test Patch, Etc.)

The test patch 2 can be produced through disposing the analysis sheet 17 at a predetermined position on the back surface side of the base material 11 of the obtained microneedle structure 10 and laminating the tape 18 so as to cover the analysis sheet 17 (installation step). A conventionally known method can be used as the lamination method. For example, the test patch 2 can be produced through placing the analysis sheet 17 on the back surface side of the base material 11 and then laminating a pressure sensitive adhesive tape 18 in which a commonly-used adhesive layer of a rubber-based pressure sensitive adhesive, an acrylic-based pressure sensitive adhesive, a silicone-based pressure sensitive adhesive, or the like is laminated on a tape base material. A drug administration patch can also be produced by a similar method.

### (Modification)

In the present embodiment, the solid composition 31 has been described as containing the water-soluble material and the water-insoluble high-molecular-weight and low-melting-point resin, but the solid composition 31 is not particularly limited, provided that it contains at least a resin. When the solid composition 31 is used as in the present embodiment, the composition does not contain a solvent, so discoloration and deformation of the base material 11 can be suppressed, which may be preferred. Furthermore, in the present embodiment, the order of the bonding step and the formation step may be reversed, and the bonding step may be performed in parallel with the formation step. That is, the recessed portion 42 may be filled with the mixture 33, and before solidification, the base material 11 may be placed on the mixture 33 to perform the bonding step, thereby obtaining the solid composition 31 with base material.

In the present embodiment, the water-insoluble high-molecular-weight and low-melting-point resin is used to form the needle-shaped portions 12 in order to readily form the hole portions 13 by removing the water-soluble material, but the method for creating the hole portions 3 is not particularly limited, provided that the aforementioned high-molecular-weight and low-melting-point resin is used. For example, the formation step may include filling the mold 52 with a particulate high-molecular-weight and low-melting-point resin or the like and sintering it at a temperature equal to or higher than the melting point of the low-melting-point resin thereby to obtain a microneedle structure having a porous structure composed of a large number of voids formed between the particles. Also in this case, when the formation step and the bonding step are carried out at the same time, the base material 11 having a layer composed of a heat-resistant resin can suppress the deformation and deterioration of the base material 11. In any case, by using the high-molecular-weight and low-melting-point resin to form the needle-shaped portions 12, there is no need to heat it at a high temperature, which results in low cost and good workability, and the substrate 11 does not deform or soften, thus allowing for a high degree of freedom in the selection of the base material 11.

In the present embodiment, the needle-shaped portions 12 are formed using a water-insoluble material in order to easily form the hole portions 13 by removing the water-soluble material, but the method for creating the needle-shaped portions 12 is not particularly limited. For example, the formation step may adopt a scheme of forming a liquid composition containing a water-soluble material, a water-insoluble material, and a solvent, evaporating the solvent, filling the projection forming recessed portions with a composition other than the solvent, and drying it to form the projecting portions. The formation step may adopt, for example, another scheme of preparing a liquid composition containing a water-soluble material and a water-insoluble material so that the viscosity is 0.1 to 1,000 mP·s, and dropping the liquid composition onto the base material 11 using a dispenser or the like, thereby forming the needle-shaped portions 12.

### EXAMPLES

The present invention will be described in more detail below with reference to Examples.

In the examples and comparative example, the weight-average molecular weight (Mw) refers to an equivalent weight-average molecular weight measured with a standard substance: polystyrene standard under the following conditions using gel permeation chromatography (GPC) (GPC measurement). Samples for GPC measurement were prepared as follows. First, 1 g of polycaprolactone (PCL) used in the examples and comparative example and 9 g of tetrahydrofuran (THE, available from FUJIFILM Wako Pure Chemical Corporation) were added to a screw tube, shaken, and completely dissolved to prepare a 10% PCL solution. Furthermore, 1 ml of the obtained solution and 9 ml of THE were dropped into a separately prepared screw tube to prepare a 1% PCL solution. This 1% PCL solution was filtered with a GD/X syringe filter (available from Whatman) and dropped into a GPC device.

### (Measurement Conditions)

Measurement device: HLC-8320 available from Tosoh Corporation
GPC columns (passing through in the following order): available from Tosoh Corporation

TSK gel super H-H
TSK gel super HM-H
TSK gel super H2000

Solvent for measurement: tetrahydrofuran
Measurement temperature: 40° C.

### Example 1

Using Labo-plastomill 4C150 (Toyo Seiki Seisaku-sho, Ltd.), 3 g of polyethylene glycol (PEG) (weight-average molecular weight of 4,000, melting point of 40° C.) as a water-soluble material and 7 g of pellet-shaped polycaprolactone (weight-average molecular weight 80,000) were heated and kneaded at 170° C. In this way, the mixture 33 was prepared. The mold for solid composition 42 composed of polydimethylsiloxane was prepared, which was formed with the recessed portion 42 having a square opening of 15 mm×15 mm and a depth of 1.5 mm. The mixture 33 was injected into the recessed portion 42 of the mold for solid composition 41 so as to fill the recessed portion 42.

The mold lid for solid composition (sheet composed of polydimethylsiloxane) 43 was placed on the mold for solid composition 41, and the surface of the solid composition 31 was flattened. This state was maintained at 3° C. for 5 minutes, and the molten mixture 33 was solidified into a solid, so it was separated from the mold for solid composition 41 to obtain the solid composition 31. Then, the pressure sensitive adhesive layer of the base material 11, which was a pressure sensitive adhesive tape (PET substrate (100 μm thick) formed with an acrylic-based pressure sensitive adhesive layer (25 μm thick) thereon), and the solid composition 31, were bonded to each other. The solid composition 31 provided with the base material 11 was thus obtained.

To carry out the formation step, the mold 52 having the projection forming recessed portions 53 was prepared. The mold 52, composed of polydimethylsiloxane, was formed with the projection forming recessed portions 53 on its surface having the recessed portion 51 as detailed below:
Shape of the projection forming recessed portions: square pyramid shape with square cross section;
Length of one side of the maximum cross section of a projection forming recessed portion: 500 μm;
Height of the projection forming recessed portions: 900 μm;
Pitch of the projection forming recessed portions: 1,000 μm;
Number of the projection forming recessed portions: 13 in vertical row, 169 in total of 13 rows;
Size of the region formed with the projection forming recessed portions: 15 mm square; and
Arrangement of the projection forming recessed portions: square grid shape.

The preliminary step was carried out through: placing the mold 52 on the lower stage 56 of a heating press machine (available from AS ONE CORPORATION, AH-1T); placing the solid composition 31 with the base material 11 on the mold 52, facing the recessed portion 51; overlapping a sheet (lid 54) composed of polydimethylsiloxane and having a 30 mm square shape from above; and pressing them at 2 MPa for 3 minutes while heating them at a lower stage setting heating temperature of 140° C. and an upper stage setting heating temperature of 140° C. of the heating press machine. After that, the main step was carried out by pressing at 4 MPa for 30 seconds in a heating state in which the temperatures of the heating press machine were retained. Furthermore, the base material 11 and the molten solid composition housed in the lid 54 and mold 52 were stored in a refrigerator at 3° C. for 5 minutes to solidify the solid composition, forming the projecting portions 32, etc. Thereafter, the base material 11 was released from the mold 52, and the base material 11 and the formed projecting portions 32, etc. were immersed in purified water at 23° C. for 24 hours to dissolve and remove the water-soluble material. After that, the base material 11 and the molded solid composition 31 were statically placed in a drying oven (30° C.) for 5 hours to evaporate water and dry, thus obtaining the microneedle structure 10.

### Example 2

The microneedle structure 10 was obtained in the same manner as in Example 1 except that 7 g of pellet-shaped polycaprolactone with a different molecular weight from that in Example 1 (weight-average molecular weight 40,000) was used as the resin constituting the needle-shaped portions 12 and the temperatures of the heating step in the formation step were all 110° C.

### Comparative Example 1

A microneedle structure was obtained in the same manner as in Example 1 except that 7 g of pellet-shaped polycaprolactone with a different molecular weight from that in Example 1 (weight-average molecular weight 10,000) was used as the resin constituting the needle-shaped portions 12, the temperatures of the heating step in the formation step were all 110° C., and the pressurization time in the preliminary step was 1 minute 30 seconds.

The microneedle structures obtained in Examples 1 and 2 and Comparative Example 1 were subjected to the following microneedle array transferability evaluation and microneedle tip strength evaluation.

### (Microneedle Array Transferability Evaluation)

In each of the examples and comparative example, projecting portions were formed by cooling the composition, and after release from the mold and before immersion in purified water, the projecting portions were observed with an optical microscope (magnification: 50× and 100×) to count the number of projecting portions remaining on the base material. The ratio of this remaining number to the total number of projecting portions in the design was calculated to determine the transfer ratio. A transfer ratio of 50% or more and 100% or less was evaluated as “A,” and a transfer ratio of 0% or more and less than 50% was evaluated as “B.” (Microneedle Tip Strength Evaluation)

The microneedle structure obtained in each of the examples and comparative example was placed on a stage with the needle-shaped portions facing up, observation with a microscope was perform to select one needle-shaped body having a sharp tip shape, and an attachment (made of iron, 2 mmp) of a measurement device (digital force gauge available from IMADA CO., LTD.) was aligned with the position of the needle-shaped portion of the needle-shaped body and was brought close to the needle-shaped portion, taking care not to allow the attachment to come into contact with adjacent needle-shaped portions. The attachment was moved up and down to a position at which it came into contact with the tip of the needle-shaped portion but no force was applied to the needle-shaped portion, and then the attachment was raised 0.1 mm from there and was thereafter lowered at a speed of 5 mm/min to start measurement of the force applied to the attachment (measurement range: 1 to 5000 mN). At that time, the measurement temperature was 23° C. and the relative humidity was 50%. At a point of time when a drop of the force was first observed on a graph on which the measured force was output, a local maximum value of the force exhibited at a position before the drop of the force was read, or a value of the force at a point of time when the fall of the attachment reached 100 μm was read if the decrease in the force was not observed until the fall of the attachment reached 100 μm. The read value was determined as the tip strength of the needle-shaped portion. When the tip strength was more than 200 mN, it was evaluated as “A,” when the tip strength was 100 to 200 mN, it was evaluated as “B,” and when the tip strength was less than 100 mN, it was evaluated as “C.” For the examples or comparative example in which the transfer ratio was less than 100% in the transferability evaluation, one of the needle-shaped portions that remained on the base material without falling off was selected and evaluated.

The evaluation results of Examples 1 and 2 and Comparative Example 1 are listed in Table 1.

TABLE 1

| | Low-melting- | | | | | | | |
|---|---|---|---|---|---|---|---|---|
| | point resin | | | | | | | |
| | | Weight- | Water-soluble | | | | | |
| | | average | material | | Heating step | | Evaluation | |
| | | molecular | | Molecular | Preliminary | Main | Transfer- | Tip |
| | Material | weight | Material | weight | step | step | ability | strength |
| Example 1 | PCL | 80,000 | PEG | 4,000 | 140 | 140 | A | A |
| Example 2 | PCL | 40,000 | PEG | 4,000 | 110 | 110 | A | B |
| Comparative | PCL | 10,000 | PEG | 4,000 | 110 | 110 | A | C |
| Example 1 | | | | | | | | |

As listed in Table 1, in Examples 1 and 2 and Comparative Example 1, the microneedle array transferability evaluation was A. On the other hand, as listed in Table 1, the microneedle tip strength evaluation was C in Comparative Example 1 in which the microneedle tip strength was less than 100 mN. From this, the strength of the needle-like portions 12 was increased when a high-molecular-weight and low-melting-point resin (weight-average molecular weight of 25,000 or more) was used.

### Example 3

A microneedle structure 10 was obtained in the same manner as in Example 1 except for the following points. The microneedle structure 10 obtained in this example does not have a base material 11.
The base material 11 was not bonded to the solid composition 31, and the lid 54 was placed directly on the solid composition 31.
Regarding the temperature and time of the heating step in the formation step, the heating temperature set for the lower stage of the heating press machine was 115° C., the heating temperature set for the upper stage was 105° C., and the time for the preliminary step was 1 minute 30 seconds (the time of the main step was not changed).
The conditions for immersing the molded solid composition 31 in purified water were 24 hours in purified water at 40° C., and the conditions for drying the molded solid composition 31 were 24 hours at 40° C.

### Example 4

A microneedle structure 10 was obtained in the same manner as in Example 3 except that 7 g of pellet-shaped polycaprolactone (weight-average molecular weight of 40,000) with a molecular weight different from that of Example 3 was used as the resin constituting the needle-shaped portions 12.

### Comparative Example 2

A microneedle structure was obtained in the same manner 3 as in Example except that g 7 of pellet-shaped polycaprolactone (weight-average molecular weight of 10,000) with a molecular weight different from that of Example 3 was used as the resin constituting the needle-shaped portions.

### Example 5

A microneedle structure 10 was obtained in the same manner as in Example 3 except that 0.5 g of ARBOCEL Ultrafine Cellulose (average particle size: 6 to 12 μm, available from Rettenmaier Japan Co., Ltd.) was added as a filler composed cellulose (water-insoluble hydrophilic resin) when preparing the mixture 33.

### Example 6

A microneedle structure 10 was obtained in the same manner as in Example 5 except that the amount of filler added was 2.0 g.

### Example 7

A microneedle structure 10 was obtained in the same manner as in Example 4 except that 0.5 g of ARBOCEL Ultrafine Cellulose (average particle size: 6 to 12 μm, available from Rettenmaier Japan Co., Ltd.) was added as a filler composed of cellulose when preparing the mixture 33.

### Example 8

A microneedle structure 10 was obtained in the same manner as in Example 7 except that the amount of filler added was 2.0 g.

### Reference Example 1

A microneedle structure 10 was obtained in the same manner as in Comparative Example 2 except that 0.5 g of ARBOCEL Ultrafine Cellulose (average particle size: 6 to 12 μm, available from Rettenmaier Japan Co., Ltd.) was added as a filler composed of cellulose when preparing the mixture 33.

### Reference Example 2

A microneedle structure 10 was obtained in the same manner as in Reference Example 1 except that the amount of filler added was 2.0 g.

The microneedle structures (needle-shaped portions without a base material) obtained in Examples 3 to 8, Comparative Example 2, and Reference Examples 1 and 2 were evaluated for water absorbability as follows.

### (Evaluation of Water Absorbability)

The weight of the sample of the needle-shaped portions before water absorption was measured. Subsequently, under an environment of 25° C., the sample was placed in a tray (available from AS ONE CORPORATION, non-charged balance dish), and 10 ml of purified water was poured in to immerse the sample. Then, the tray was placed under a reduced pressure environment of 0.09 MPa for 1 hour to allow water to penetrate into the interior of the porous structure. Subsequently, the sample was taken out from the tray, and water droplets attached to the surface were removed. Specifically, water droplets on the surfaces of the needle-shaped portions on the side formed with needles were removed by blowing them off with an air blow gun. In addition, water droplets on the surfaces of the base portion side of the needle-shaped portions were removed through placing the base portion on a glass plate and allowing the needle-shaped portions' own weight to push the water droplets around the base portion, and after statically placing it for 5 seconds, the needle-shaped portions were picked up from the glass plate. After that, weight measurement was performed for the sample after water absorption. Then, the water absorption ratio (ratio of absorbed water to the sample's own weight) was obtained using an equation below.

$$Water absorption ratio ( \% ) = ( weight of sample after water absorption - weight of sample before water absorption ) \div weight of sample before water absorption \times 100$$

The evaluation results are listed in Table 2. In Table 2, the “additive amount” of the water-insoluble hydrophilic resin is the ratio of the mass of the water-insoluble hydrophilic resin to the total mass of the low-melting point resin and the water-soluble resin, expressed as a percentage.

TABLE 2

| | Low-melting- | | | | | | |
|---|---|---|---|---|---|---|---|
| | point resin | | | | Water-insoluble | | |
| | | Weight- | Water-soluble | | hydrophilic group- | | Evaluation |
| | | average | material | | containing resin | | Water |
| | | molecular | | Molecular | | Additive | absorption |
| | Material | weight | Material | weight | Material | amount | ratio |
| Example 3 | PCL | 80,000 | PEG | 4,000 | — | — | 9.1% |
| Example 4 | PCL | 40,000 | PEG | 4,000 | — | — | 9.2% |
| Comparative | PCL | 10,000 | PEG | 4,000 | — | — | 7.5% |
| Example 2 | | | | | | | |
| Example 5 | PCL | 80,000 | PEG | 4,000 | Cellulose fiber | 5% | 21.6% |
| Example 6 | PCL | 80,000 | PEG | 4,000 | Cellulose fiber | 20% | 29.7% |
| Example 7 | PCL | 40,000 | PEG | 4,000 | Cellulose fiber | 5% | 15.4% |
| Example 8 | PCL | 40,000 | PEG | 4,000 | Cellulose fiber | 20% | 24.9% |
| Reference | PCL | 10,000 | PEG | 4,000 | Cellulose fiber | 5% | 17.7% |
| Example 1 | | | | | | | |
| Reference | PCL | 10,000 | PEG | 4,000 | Cellulose fiber | 20% | 26.0% |
| Example 2 | | | | | | | |

The water absorption ratios of Example 3 and Example 2 were higher than that of Comparative Example 2, and it has been confirmed that the water absorption ratio increases as the molecular weight of the low-melting point resin increases. Furthermore, in Examples 5 and 6 and Examples 7 and 8, the water absorption ratio was significantly increased by including cellulose, a water-insoluble hydrophilic resin, compared to Examples 3 and 4 having the same molecular weight of the low-melting-point resin. Even in these cases, Examples 5 and 6, in which the weight-average molecular weight of the low-melting-point resin was 80,000, tended to have a higher water absorption ratio than Examples 7 and 8 and Reference Examples 1 and 2 containing the same amount of cellulose.

### INDUSTRIAL APPLICABILITY

The microneedle structure of the present invention can be used as a test patch, for example, by placing an analysis sheet on the back surface side and laminating it with a tape.

### DESCRIPTION OF REFERENCE NUMERALS

10 Microneedle structure
11 Base material
12 Needle-shaped portion
13 Hole portion
14 Base portion
15 Through-hole
16 Adhesive layer
31 Solid composition
32 Projecting portion
33 Mixture

1. A microneedle structure comprising a needle-shaped portion having an interior formed with a hole portion,
the needle-shaped portion containing a low-melting-point resin having a weight-average molecular weight of 25,000 or more and a melting point of 130° C. or lower.

2. The microneedle structure according to claim 1, wherein the needle-shaped portion contains a water-insoluble hydrophilic resin.

3. The microneedle structure according to claim 2, wherein the water-insoluble hydrophilic resin is a water-insoluble polysaccharide.

4. The microneedle structure according to claim 1,
wherein the needle-shaped portion is formed with a porous structure.

5. The microneedle structure according to claim 4, wherein the needle-shaped portion has a base portion, and a water absorption ratio of the needle-shaped portion measured by a testing method below in a state in which the needle-shaped portion is composed only of the porous structure is 8.5% or more.
(Testing method)
The needle-shaped portion is immersed in 10 ml of purified water under an environment of 25° C.
The needle-shaped portion in an immersed state is placed under a reduced pressure environment of 0.09 MPa for 1 hour to allow water to penetrate into the interior of the porous structure.
Subsequently, the needle-shaped portion is taken out from the purified water, and water droplets attached to the surface are removed. Water droplets on the surface of the needle-shaped portion on a side formed with a needle are removed by blowing them off with an air blow gun, and water droplets on the surface of the base portion side of the needle-shaped portion are removed through placing the base portion on a glass plate and allowing the needle-shaped portion's own weight to push the water droplets around the base portion, and after statically placing it for 5 seconds, the needle-shaped portion is picked up from the glass plate. Then, weight measurement is performed for a sample after water absorption. The water absorption ratio (ratio of absorbed water to the sample's own weight) is obtained using an equation below.
$$Water absorption ratio ( \% ) = ( weight of sample after water absorption - weight of sample before water absorption ) \div weight of sample before water absorption \times 100$$

6. The microneedle structure according to claim 1, wherein the needle-shaped portion contains a filler.
